(19) United States
(12) Patent Application Publication
(10) Pub. No.: US 2005/0065733 A1
(43) Pub. Date: Mar. 24, 2005

(54) Visualization of databases

(76) Inventors: Paul Caron, Malden, MA (US); Brian Hare, Cambridge, MA (US); Brian McClain, Watertown, MA (US); W. Patrick Walters, Westborough, MA (US); Trevor Kramer, Puyallup, WA (US)

Correspondence Address: FISH & RICHARDSON PC, 225 FRANKLIN ST, BOSTON, MA, 02110, US

(21) Appl. No.: 10/914,342

(22) Filed: Aug. 9, 2004

Related U.S. Application Data
(60) Provisional application No. 60/493,682, filed on Aug. 8, 2003.

(51) Int. Cl. G06F 19/00; G01N 33/48; G01N 33/50; G01N 31/00; G03C 8/00

(52) U.S. Cl. 702/19; 702/22; 705/2

(57) ABSTRACT

This invention relates to computer-based methods, systems, and databases for visualizing chemical structure relatedness.

### CROSS REFERENCE TO RELATED APPLICATIONS

This application claims the benefit of U.S. Provisional Application No. 60/493,682, filed on Aug. 8, 2003, the contents of which are hereby incorporated by reference in its entirety.

### TECHNICAL FIELD

This invention relates to computer-based methods, systems, and databases for visualizing chemical structure relatedness.

### BACKGROUND

A recent estimate has placed the number of organic chemical compounds with a molecular weight less than 800 at about 10²⁰⁴. Thus, many of the known (e.g., disclosed in scientific journals or patents) chemical compounds that have been isolated from natural sources and/or synthesized in the world's laboratories have now been compiled in centralized and searchable computer database systems and additional compounds are continually being compiled. The path to fruitful research often begins with an understanding of what is already known and what is as yet to be discovered. With regard to chemical compounds, querying one or more of the above database systems can typically answer the former question. However, these database systems cannot provide data that does not yet exist. Thus, there is a need for a system that can assist a user with identifying new compounds within the context of the existing ones.

### SUMMARY

This invention relates to computer-based methods, computer systems, and databases for visualizing chemical structure relatedness. The computer-based methods are performed utilizing computer systems or parts thereof, including for example, those described herein.

In one aspect, this invention relates to a method for visualizing a database of chemical structures from the patent literature. The method includes mapping a database of chemical structures from patent literature documents, wherein each of the chemical structures is displayed on a map as a discrete marker, and the intervening space between the discrete markers is displayed on the map as a continuum that visually contrasts with the discrete markers.

Embodiments may include one or more of the following features.

The structures can be explicitly disclosed and/or claimed in U.S. patents and/or U.S. patent application publications, and/or PCT application publications, and/or non-U.S. patents and/or non-U.S. patent application publications.

The structures can be implicitly disclosed and/or claimed in U.S. patents and/or U.S. patent application publications, and/or PCT application publications, and/or non-U.S. patents and/or non-U.S. patent application publications.

The mapping can be carried out according to a user-defined similarity parameter, e.g., structural similarity.

The map can be a linear map or a non-linear map. The distance between any two discrete markers on the linear or non-linear map can be representative of the similarity or dissimilarity between the corresponding chemical structures.

The database of chemical structures can further include one or more data fields related to each of the chemical structures, e.g., biological assay data related to one or more biological targets, a medical indication, a physical property, a key word, a patent assignee, a patent issue date, a patent application filing date, an inventor name, or inventory data.

In another aspect, this invention relates to a method for generating a database of compounds that are outside of an original database. The method includes: (a) mapping a database of original chemical structures, wherein each of the original chemical structures is displayed as a discrete marker, and the intervening space between the discrete markers is displayed as a continuum that visually contrasts with the discrete markers; (b) mapping a database of query chemical structures, wherein each of the query chemical structures is displayed as a differentiable discrete marker that is differentiable from the discrete markers corresponding to the database of original chemical structures; and (c) determining the degree of similarity between the query chemical structures and the original chemical structures.

Embodiments may include one or more of the following features.

The original chemical structures can be from patent literature documents. The structures can be explicitly disclosed and/or claimed in U.S. patents and/or U.S. patent application publications, and/or PCT application publications, and/or non-U.S. patents and/or non-U.S. patent application publications. The structures can be implicitly disclosed and/or claimed in U.S. patents and/or U.S. patent application publications, and/or PCT application publications, and/or non-U.S. patents and/or non-U.S. patent application publications.

The mapping can be carried out according to a user-defined similarity parameter, e.g., structural similarity.

The map can be a linear map or a non-linear map. The distance between any two discrete markers on the linear or non-linear map can be representative of the similarity or dissimilarity between the corresponding chemical structures.

The database of chemical structures can further include one or more data fields related to each of the chemical structures, e.g., biological assay data related to one or more biological targets, a medical indication, a physical property, a key word, a patent assignee, a patent issue date, a patent application filing date, an inventor name, or inventory data.

Steps (a) and (b) can be performed simultaneously, and the discrete markers corresponding to the original chemical structures, the intervening space between the discrete markers, and the differentiable discrete markers corresponding to the query chemical structures can be displayed on a map.

Steps (a) and (b) can be performed at different times. Embodiments may also include methods that further include the steps of (i) displaying the discrete markers corresponding to the original chemical structures and the intervening space between the discrete markers on a first map; (ii) displaying the differentiable discrete markers corresponding to the query chemical structures on a second map; and (iii) overlaying the first and second maps. Embodiments may also include methods that further include displaying the discrete markers corresponding to the original chemical structures and the intervening space between the discrete markers on a map that is automatically updated with the differentiable discrete markers corresponding to the query chemical structures once step (b) is performed.

The query chemical structure can be a unique query chemical structure, e.g., a de novo structure.

The method can further include the step of providing a database of original chemical structures structures, which may further include representing the chemical structures in binary form or in the form of binary fingerprints.

The method can further include the step of providing a database of query chemical structures structures, which may further include representing the chemical structures in binary form or in the form of binary fingerprints.

In a further aspect, this invention relates to a method for generating a database of compounds that are outside of an original database. The method includes: (a) providing a database of original chemical structures; (b) mapping the database of original chemical structures, wherein each of the chemical structures is displayed on a map as a discrete marker and the intervening space between the markers is displayed on the map as a continuum that visually contrasts with the plurality of discrete markers; (c) providing a database of one or more query chemical structures; (d) mapping the database of query chemical structures, wherein each query chemical structure is displayed on the map from step (b) as a differentiable discrete marker that is differentiable from the discrete markers corresponding to the original chemical structures; (e) determining the degree of similarity between the query chemical structures and the original chemical structures; (f) providing a database of one or more modified query chemical structures, wherein each structure corresponds to a query chemical structure from step (c) having a modification, and wherein the modification is chosen so that the modified query chemical structure is less similar to a comparative subset of original chemical structures than the query chemical structure before the modification; (g) mapping the database of modified query chemical structures, wherein each modified query structure is displayed on the map from step (b) or step (d) as a differentiable discrete marker that is differentiable from the discrete markers corresponding to the original chemical structures and from the differentiable discrete markers corresponding to the query chemical structures; and (h) determining the degree of similarity between the modified query chemical structures and the comparative subset of original chemical structures from step (f).

Embodiments may include one or more of the following features.

Steps (c)-(e) can be repeated until a query chemical structure is found that is unique with respect to the original chemical structures.

Steps (f)-(h) can be performed on a query chemical structure that is substantially similar to an original chemical structure.

Steps (f)-(h) can be repeated. Embodiments may also include methods in which steps (f)-(h) can be repeated using the same query chemical structure and a different modification; steps (f)-(h) can be repeated using a different query chemical structure and the same modification; or steps (f)-(h) are repeated using a different query chemical structure and a different modification.

The original chemical structures can be from patent literature documents. The structures can be explicitly disclosed and/or claimed in U.S. patents and/or U.S. patent application publications, and/or PCT application publications, and/or non-U.S. patents and/or non-U.S. patent application publications. The structures can be implicitly disclosed and/or claimed in U.S. patents and/or U.S. patent application publications, and/or PCT application publications, and/or non-U.S. patents and/or non-U.S. patent application publications.

Steps (a), (c), and (f) can further include representing the chemical structures in binary form, or in the form of binary fingerprints.

The database of chemical structures can further include one or more data fields related to each of the chemical structures, e.g., biological assay data related to one or more biological targets, a medical indication, a physical property, a key word, a patent assignee, a patent issue date, a patent application filing date, an inventor name, or inventory data.

The mapping can be carried out according to a user-defined similarity parameter, e.g., structural similarity.

The map can be a linear map or a non-linear map. The distance between any two discrete markers on the linear or non-linear map can be representative of the similarity or dissimilarity between the corresponding chemical structures.

The query chemical structure can be a unique query chemical structure, e.g., a de novo structure.

In one aspect, this invention relates to a method for generating a database of compounds that are outside of an original database. The method includes: (a) providing a database of original chemical structures; (b) mapping the database of original chemical structures, wherein each of the chemical structures is displayed on a map as a discrete marker and the intervening space between the markers is displayed on the map as a continuum that visually contrasts with the plurality of discrete markers; (c) providing a database of one or more de novo chemical structures; (d) mapping the database of de novo chemical structures, wherein each de novo chemical structure is displayed on the map from step (b) as a differentiable discrete marker that is differentiable from the discrete markers corresponding to the original chemical structures; (e) determining the degree of similarity between the de novo chemical structures and the original chemical structures; and (f) evaluating the number of discrete markers in the intervening space continuum. Other embodiments may include one or more of the features delineated above.

In another aspect, this invention relates to a database generated by: (a) providing a database of original chemical structures; (b) mapping the database of original chemical structures, wherein each of the chemical structures is displayed on a map as a discrete marker and the intervening space between the markers is displayed on the map as a continuum that visually contrasts with the plurality of discrete markers; (c) providing a database of one or more unique query chemical structures, wherein each unique query chemical structure is unique with respect to the original chemical structures; (d) mapping the database of unique query chemical structures, wherein each unique chemical structure is displayed within the intervening space continuum on the map from step (b) as a differentiable discrete marker that is differentiable from the discrete markers corresponding to the original chemical structures; (e) determining the degree of similarity between the unique query chemical structures and the original chemical structures; (f) providing a database of one or more modified unique query chemical structures, wherein each modified structure corresponds to a unique query chemical structure from step (c) having a modification, and wherein the modification is chosen so that the modified unique query chemical structure is less similar to a comparative subset of original chemical structures than the unique query chemical structure was before the modification; (g) mapping the modified unique query chemical structures, wherein each modified unique query chemical structure is displayed on the map from step (b) or step (d) as a differentiable discrete marker that is differentiable from the discrete markers corresponding to the original chemical structures and from the discrete markers corresponding to the unique query chemical structures; and (h) determining the degree of similarity between the modified unique query chemical structures and the comparative subset of original chemical structures from step (f). Other embodiments may include one or more of the features delineated above.

In a further aspect, this invention features a method for designing a drug candidate. The method includes: (a) providing a database of original chemical structures; (b) mapping the database of original chemical structures, wherein each of the chemical structures is displayed on a map as a discrete marker and the intervening space between the markers is displayed on the map as a continuum that visually contrasts with the plurality of discrete markers; (c) providing a database of one or more de novo chemical structures; (d) mapping the database of de novo chemical structures, wherein each de novo chemical structure is displayed on the map from step (b) as a differentiable discrete marker that is differentiable from the discrete markers corresponding to the original chemical structures; (e) determining the degree of similarity between the de novo chemical structures and the original chemical structures; (f) evaluating the number of differentiable discrete markers located in the intervening space continuum.

(g) selecting a chemical structure corresponding to a differentiable discrete marker located in the intervening space continuum; and (h) subjecting the chemical structure to computer-aided drug design methods.

Embodiments may include one or more of the following features.

The method may further include synthesizing the compound corresponding to the structure selected in step (g).

The method may further include evaluating the compound's ability to modulate a target through in vivo and/or in vitro methods.

Other embodiments may include one or more of the features delineated above.

In one aspect, this invention relates to a method for visualizing the relationship of a drug candidate chemical structure to structures in a database. The method includes: (a) providing a database of original chemical structures; (b) mapping the database of original chemical structures, wherein each of the chemical structures is displayed on a map as a discrete marker and the intervening space between the markers is displayed on the map as a continuum that visually contrasts with the plurality of discrete markers; (c) providing a database of one or more drug candidate chemical structures; (d) mapping the database of drug candidate chemical structures, wherein each drug candidate chemical structure is displayed on the map from step (b) as a differentiable discrete marker that is differentiable from the discrete markers corresponding to the original chemical structures; and (e) evaluating the number of differentiable discrete markers located in the intervening space continuum.

Embodiments may include one or more of the following features.

The method can further include: (f) selecting a drug candidate chemical structure corresponding to a differentiable discrete marker located in the intervening space continuum; (g) measuring the distance between the differentiable discrete marker selected in step (f) and each of the discrete markers corresponding to the original chemical structures; (h) determining the discrete marker that is closest in linear distance to the differentiable discrete marker selected in step (f); (i) comparing the structure corresponding to the discrete marker determined in step (h) with the structure of the drug candidate structure corresponding to the differentiable discrete marker selected in step (f); (j) determining the discrete marker that is next closest in linear distance to the differentiable discrete marker selected in step (f); and (k) comparing the structure corresponding to the discrete marker determined in step (j) with the structure of the drug candidate structure corresponding to the differentiable discrete marker selected in step (f).

Steps (j) and (k) can be repeated.

Other embodiments may include one or more of the features delineated above.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is an overview of the chemical structure relatedness methods and corresponding maps.

FIG. 2 is a block diagram of a programmable processing system.

FIG. 3A is a map of a database of original chemical structures based on patent literature and color-coded by assignee.

FIG. 3B is a map of a database of original chemical structures to which has been added a database of query chemical structures.

FIG. 3C is a map of a database of original chemical structures to which has been added a database of query chemical structures based on patent literature and a database of query chemical structures generated with a combinatorial library enumeration method.

FIGS. 4A, 4B, 4C, and 4D show changes over time in a map of a database of chemical structures (DCS) based on structures from patent literature.

Like reference symbols in the various drawings indicate like elements.

### DETAILED DESCRIPTION

The term “chemical structure” or “structure” as used herein refers to any visual or textual representation of a chemical entity that identifies or encodes its constituent atoms and their connectivity to one another (e.g., Kekule structure, shorthand structure, Lewis structure, ball and stick structure, space-filling model, SMILES string, MOLFILE, or IUPAC name). The representation may omit hydrogen atoms for clarity or computational simplicity.

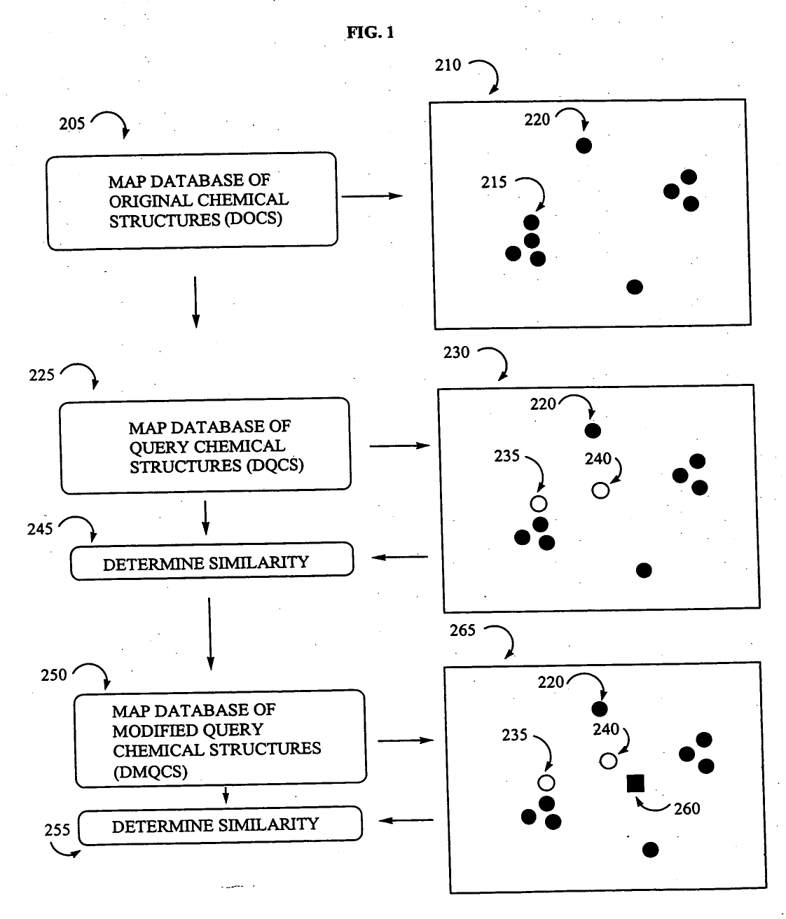

An overview of the chemical structure relatedness methods and corresponding maps is shown in FIG. 1.

Databases

The database can be any database known in the art, e.g., an Oracle® system, MYSQL, DB2, or flat file (text file) representation. As used herein, the term “database of chemical structures” (DCS) refers generally to any collection of chemical structures (e.g., A, B, C . . . ) that is to be mapped and evaluated according to the methods described herein for chemical structure relatedness, e.g., relatedness of the structures to one another (e.g., A to B, B to C, . . . ), or relatedness of the structures to one or more different databases of chemical structures (e.g., A, B, and C to DCS1, A, B; and C to DCS2, . . . ). Evaluation of chemical structure relatedness can include, e.g., determining the degree of similarity/dissimilarity that exists within a group of chemical structures.

The DCS can include any collection of chemical structures from any source, e.g., any printed publication or any public or proprietary structure databases, which may be of interest or assistance to a user of the method, e.g., as a reference set of structures for identifying de novo compounds. In one preferred embodiment, at least one structure and up to 100%, e.g., any number % between 1-100 inclusive, of the chemical structures in the DCS depict chemical entities from a proprietary database, e.g., a corporate, commercial, or academic research database. In another preferred embodiment, at least one structure and up to 100%, e.g., any number % between 1-100 inclusive, of the chemical structures in the DCS depict chemical entities that are explicitly and/or implicitly disclosed and/or claimed in one or more patent literature documents. Patent literature documents can include, without limitation, U.S. patents, U.S. patent application publications, PCT application publications, non-U.S. patents, and non-U.S. patent application publications. An “explicitly disclosed and/or claimed chemical entity” refers to an entity that is identified either textually, e.g., “1,4-dichlorobenzene,” or pictorially, e.g., as a Kekule structure, shorthand structure, Lewis structure, ball and stick structure, or space-filling model, in the specification, drawings, appendix, or claims of a patent or patent application publication. An “implicitly disclosed and/or claimed chemical entity” refers to an entity corresponding to an unrecited species that is covered by a particular chemical structure genus description (e.g., a chemical structure formula having one or more variable atom or functional group positions defined as selected from a listing of possible atoms or functional groups) disclosed and/or claimed in a patent or patent application publication. In certain embodiments, one or more structures corresponding to implicitly disclosed and/or claimed chemical entities can be enumerated, e.g., manually by a user via visual inspection of the genus and/or by computer-assisted methods, e.g., a library (e.g., a combinatorial library) enumeration program, e.g., Reaction Toolkit (Daylight Chemical Information Systems, Mission Viejo, Calif., http://www.daylight.com); Project Library (MDL Information Systems, San Leandro, Calif. http://www.mdli.com); Accord CombiChem (Accelrys, San Diego, Calif. http://www.accelrys.com).

As used herein, the term “database of original chemical structures” (DOCS) refers to the first in a series of two or more databases to be mapped and evaluated according to the methods described herein. The DOCS can also include any collection of chemical structures from any source, e.g., any printed publication or any public or proprietary structure databases, which may be of interest or assistance to a user of the method, e.g., as a reference set of structures for identifying de novo compounds. In one preferred embodiment, at least one structure and up to 100%, e.g., any number % between 1-100 inclusive, of the chemical structures in the DCS depict chemical entities from a proprietary database, e.g., a corporate, commerical, or academic research database. In another preferred embodiment, at least one structure and up to 100%, e.g., any number % between 1-100 inclusive, of the chemical structures in the DOCS depict chemical entities that are explicitly and/or implicitly disclosed and/or claimed in one or more patent literature documents. Patent literature documents can include, without limitation, U.S. patents, U.S. patent application publications, PCT application publications, non-U.S. patents, and non-U.S. patent application publications. In another preferred embodiment, at least one structure and up to 100%, e.g., any number % between 1-100 inclusive, of the chemical structures in the DCS depict chemical entities from a proprietary database, e.g., a corporate, commercial or academic research database. An “explicitly disclosed and/or claimed chemical entity” refers to an entity that is identified either textually, e.g., “1,4-dichlorobenzene,” (using nomenclature or naming roles known to one of ordinary skill in the art; e.g., IUPAC) or pictorially, e.g., as a Kekule structure, shorthand structure, Lewis structure, ball and stick structure, or space-filling model, in the specification, drawings, appendix, or claims of a patent or patent application publication. An “implicitly disclosed and/or claimed chemical entity” refers to an entity corresponding to an unrecited species that is covered by a particular chemical structure genus description (e.g., a chemical structure formula having one or more variable atom or functional group positions defined as selected from a listing of possible atoms or functional groups) disclosed and/or claimed in a patent or patent application publication. In certain embodiments, one or more structures corresponding to implicitly disclosed and/or claimed chemical entities can be enumerated e.g., manually by a user via visual inspection of the genus and/or by computer-assisted methods, e.g., library (e.g., a combinatorial library) enumeration program, e.g., Reaction Toolkit (Daylight Chemical Information Systems, Mission Viejo, Calif., http://www.daylight.com); Project Library (MDL Information Systems, San Leandro, Calif., http://www.mdli.com); Accord CombiChem (Accelrys, San Diego, Calif., http://www.accelrys.com).

As used herein, the term “database of query chemical structures” (DQCS) refers to a collection of one or more query chemical structures that a user wishes to evaluate (i) for relatedness to one another and/or (ii) for relatedness to structures belonging to a previously mapped database of chemical structures, e.g., a DOCS. The query chemical structures that comprise the DQCS may be selected as desired. In one embodiment, query chemical structures may be selected at random, i.e., without a priori knowledge of their relatedness to the structures belonging to the previously mapped database of chemical structures. Thus, in this embodiment, the DQCS may contain one or more chemical structures that are identical to one or more of the structures belonging to the previously mapped database of chemical structures. In another embodiment, query chemical structures may be selected so that at least one and up to 100% of the query chemical structures in the DQCS are “unique” with respect to each of the chemical structures belonging to the previously mapped database of chemical structures. As used herein, a unique query chemical structure possesses one or more structural attributes, e.g., presence of a maximim or minimum number of heteroatoms (nitrogen, oxygen, sulfur, phosphorus, fluorine, etc.), notable or important electronic configurations and atom types (doubly bonded nitrogen, aromatic carbon, etc.), presence of a particular functional group or groups (hydroxyl, amino, carboxy, etc.) that render it non-identical with respect to the chemical structures belonging to the previously mapped database of chemical structures. Briefly, if a DOCS contains structures J, K, L, and M, a DQCS may contain, e.g., K, O, and P (1 identical, 2 unique) or O, P, and Q (all unique).

As used herein, the term “database of modified query chemical structures” (DMQCS) refers to a collection of one or more modified query chemical structures that a user wishes to evaluate (i) for relatedness to one another and/or (ii) for relatedness to structures belonging to a previously mapped DQCS and/or (iii) for relatedness to one or more other previously mapped databases of chemical structures, e.g., a DOCS. A “modified query chemical structure” is based on a query chemical structure from a previously mapped DQCS and has one or more modifications (e.g., the deletion or addition of a chemical bond, atom or collective group of bonded atoms; or the replacement of one atom by another) that render it non-identical with respect to the chemical structures belonging to the previously mapped databases of chemical structures, e.g., the DQCS and the DOCS. Following the example above, the query chemical structure used as the basis for the modified query chemical structure can either be K, the identical structure in the DQCS, or O, P, and Q, the unique structures in the DQSC. Thus, if a DOCS contains structures J, K, L, and M, a DQCS may contain, e.g., K, O, and P (1 identical, 2 unique) or O, P, and Q (all unique), and a DMQCS may be based on K, O, P or Q and have one or more modifications as described above. Thus, a DMQCS chemical structure can be, e.g., any derivative, analog, or isomer of any DQCS chemical structure, e.g., K′, K″, K′″ . . . ; or O′, O″, O′″ . . . ; or P′, P″, P′″ . . . ; or Q′, Q″, Q′″ . . . .

The user may select structures to populate the above databases using any method of structural representation known in the art, e.g., Simplified Molecular Input Line Entry Specification (SMILES; www.daylight.com). The structures may be created and inputted “from scratch” using any chemical drawing tool known in the art, e.g., MarvinSketch (Chemaxon, Ltd., Budapest, Hungary, www.chemaxon.com/marvin) or other chemical drawing software packages, e.g., ChemDraw® (Cambridgesoft, Cambridge, Mass.) or ISIS™ Draw (MDL Information Systems, Inc. San Leandro, Calif.). Alternatively, the original representation can be imported as an electronic file e.g., from an electronic mail message or report, created or drawn by other users at other times, or stored and recalled from a memory storage device.

In certain embodiments, one or more structures within the above databases may have one or more associated data fields. The data field can be, e.g., without limitation, biological data (e.g., bioefficacy, toxicology, binding data, assay data related to one or more targets, medical indications, e.g., diseases or disorders); source or inventory data; physicochemical data; assignees of U.S. and non U.S. patents; publication date of printed publication data; submission date of printed publication data; priority, filing, issue, and expiration dates of U.S. and non-U.S. patents and publication dates for U.S., PCT, and non-U.S. patents applications; inventor names; authors of printed publications; inventor addresses; addresses for authors of printed publications; key word or words; indication of use; mechanism of action; organism against which a compound (corresponding to a particular structure in the database) is tested; corporate entity associated with the patent/publication; pharmacological data; physical property characteristic of a compound; corresponding genomic data (structure interactions with product of a particular gene); international and U.S. national classification codes; intellectually assigned taxonomies and ontologies; clinical trial data; pre-clinical safety and animal studies; cited and citing references for patents and printed publications; primary examiner; attorney, agent or firm; title of document; abstract of document; claims of patent document; detailed description of invention; family data; extension data; expected expiry date; legal status data; or examiner field of search.

Mapping the Databases

As used herein, the term “mapping” refers to the creation and generation of a visual output that conveys to the user a qualitative and/or quantitative measure of the relatedness, e.g., degree of similarity, that exists among a particular collection of chemical structures. The visual output can be in the form of a linear or nonlinear map. In a preferred embodiment, the visual output is a nonlinear map, in which the map has a point corresponding to each structure in the collection and the distances between any two points is representative of the degree of similarity/dissimilarity between the two structures, e.g., compounds that are more similar are shown as points that are closer together on the map than points that represent compounds that are more dissimilar.

The creation and generation of nonlinear maps is within the skill of the art.

Conventional chemical structures, e.g., a Kekule structure, shorthand structure, Lewis structure, ball and stick structure, or space-filling model, can be represented as “high dimensional data” data suitable for nonlinear mapping techniques. For example, chemical structures can be represented in binary form, in which the structure is represented as a bitmap. In one embodiment, the chemical structure is encoded using substructure keys wherein each bit is used to indicate the presence or absence (or potential presence or absence) of a particular structural feature or pattern. Features can include, e.g., without limitation, the presence of a maximim or minimum number of heteroatoms (nitrogen, oxygen, sulfur, phosphorus, fluorine, etc.), notable or important electronic configurations and atom types (doubly bonded nitrogen, aromatic carbon, etc.), or presence of a particular functional group or groups (hydroxyl, amino, carboxy, etc.) In another embodiment, chemical structures may be codified in the form of “fingerprints,” e.g., binary fingerprints. Encoding of chemical structures as fingerprints, can be performed using software or computer programs known in the art including, e.g., ISIS (MDL Information Systems, San Leandro, Calif., http://www.mdli.com); BCI Fingerprint Toolkit (Barnard Chemical Information Systems, Sheffield, UK, http://www.bci.gb.com); Daylight Fingerprint Toolkit (Daylight Chemical Information Systems, Mission Viejo, Calif. http://www.daylight.com), or alternatively, any proprietary software or computer program that is suitable for carrying out similar functions. In still another embodiment, chemical structures may be codified in the form of a pharmacophore fingerprint, which is a binary bitstring containing information about one or more pharmacophores present in a structure, e.g., a hydrogen bond donor or acceptor or a formal positive or negative charge. A pharmacophore fingerprint can be generated e.g., from a one, two, or three-dimensional representation of a structure, using a program, e.g. Chem-X software (Accelrys, San Diego, Calif. http//www.accelrys.com) or “PharmPrint” (McGregor et al. J. Chem. Inform. Sci. 2000, 40, 569 and references therein), that assigns pharmacophoric groups to atoms in the structure, rotates bonds to generate multiple conformations, and builds the fingerprint by measuring distances between pharmacophoric groups.

These “higher dimensional” representations of the chemical structures, along with a user-defined similarity parameter, e.g., structural similarity, may in turn be embedded in meaningful lower dimensional space, i.e. mapped, using any algorithm, e.g., one that is capable of reducing a high-dimensional input data set of objects to an lower-dimensional output data set of objects, such that the degree of similarity among the objects in the input data set is conserved in the form of a distance function in the output data set, e.g., a given object will be more proximal to other similar objects than it will be to less similar objects. High dimensional spaces can be inherently difficult to understand, and in general, their structure cannot be easily extracted with conventional graphical techniques.

The dimensional lowering or reducing technique may be a linear technique, e.g., Principal Component Analysis (PCA), in which the data set is projected from high dimensional space onto a line, plane, or three-dimensional coordinate system. In PCA, a data matrix (high dimensional data) is broken down via principal component transformation into a scores matrix (i.e., new coordinates in reduced dimensional space) and a loading matrix, which defines the transformation and can be used to add new objects to the reduced space. PCA is described in e.g., Hotelling, H., J. Edu. Psychol. 1933, 24, 417-441; 498-520.

In preferred embodiments, the dimensional lowering or reducing technique may be a non-linear technique, e.g., Multidimensional Scaling (MDS). In MDS, a set of objects, k, having rᵢⱼ relationships (i.e., high dimensional data) is projected in reduced dimensional space as a set of images, x, such that their Euclidean distances dᵢⱼ=∥yᵢ−yⱼ∥ approximates as closely as possible the the corresponding values rᵢⱼ. This projection can be carried out in iterative fashion by minimizing an error function, e.g., Kruskal's stress function, which measures the difference between the original, rᵢⱼ, and projected, dᵢⱼ, distances of the original and projected vector sets, respectively. In one embodiment, dimensionality reduction can be performed interatively by (i) computing relationships, rᵢⱼ; (ii) initializing images (e.g. chemical structures), xᵢ; (iii) computing the distances of the images, dᵢⱼ, and the value of the error function; (iv) computing a new configuration of the images xᵢ; and (v) repeating steps (iii) and (iv) until the error is minimized. In another embodiment, dimensionality reduction can be performed interatively by (i) placing objects (e.g., chemical structures) on a map; (ii) selecting a subset of the objects, preferably a pair of objects, in which the selected subset includes associated relationships between the objects in the subset; (iii) revising the distances between the objects on the map based on the relationships between the objects and the distances; and (iv) repeating steps (ii) and (iii) for additional subsets of objects from the set of objects. In a preferred embodiment, dimensionality reduction can be performed by (i) non-linearly mapping a sample of points from a multidimensional data set; (ii) determining a nonlinear function for the non-linearly mapped sample of points; and (iii) mapping additional points using the nonlinear function. In another preferred embodiment, dimensionality reduction can be performed by (i) creating a set of locally defined neural networks trained according to a mapping of a subset of the n-dimensional input patterns into an m-dimensional output space (i.e., m<n); and (ii) mapping additional n-dimensional input patterns using the locally defined networks. Schiffman, Reynolds, and Young Introduction to Multidimensional Scaling, Academic Press, New York, 1981, WO/01/71624, U.S. Pat. No. 6,453,246, U.S. Pat. No. 6,571,227, WO/99/57686, WO/00/67148, and US 2002/0099675 relate to the above methods for dimensionality reduction. Other dimensionality reduction techniques may be used, e.g., Local Linear Embedding (LLE), and Isometric Mapping (ISOMAP), which substitute an estimated geodesic distance for the Euclidean distance in MDS. These techniques are described in e.g., Roweis, S. T., Saul, L. K. Science 2000, 290, 2323-2326 and Tenenbaum, J. B., de Silva, V., Langford, J. C., Science, 2000, 290, 2319-2323. In a preferred embodiment, dimensionality reduction can be performed with VxOrd, which uses a force-directed graph layout algorithm to transform a set of pairwise similarities into a set of coordinates where the distance between points is proportional to the similarity values. VxOrd is described in Kim, S. K., Lund, J., Kiraly, M., Duke, K., Jiang, M., Stuart, J. M., Eizinger, A., Wylie, B. N., and Davidson, G. S. Science; 2001; 293, 2087-2092.

A preferred visualization output or map is shown in FIG. 1, e.g., 210. Each structure is represented as a discrete marker (solid circles), e.g., 215 and 220, and the intervening space between the discrete markers is visualized or displayed as a continuum (e.g., “white space” or “open space”) that visually contrasts with the discrete markers. The open space may be thought of in the context of this invention, as the space where unique compounds related to, e.g. 215 and 220, would appear on the map. If the discrete markers in 210 corresponded to the structures of compounds in a patent portfolio, the open space can be thought of as a map location where a compound not in the patent portfolio would appear. This space can be referred to as “exploratory space”. The structure cluster near 215 can indicate that there is a lesser degree of exploratory space with respect to this structure than there is with 220, which does not have closely neighboring discrete markers.

Maps 230 and 265 in FIG. 1 show the results of the progressive addition of the query chemical structures from the DQCS and a modified query chemical structure from the DMQCS, which are displayed to the user as differentiable, i.e. visually distinguishable, discrete markers, e.g., 235, 240, and 260. A query chemical structure can either be identical or unique with respect to the DOCS chemical structure. In map 230, the structure corresponding to marker 235 is identical to the structure corresponding to marker 215 in map 210. As a result, that particular point on the map now appears to the user as an open circle to match the appearance of the other DQCS structure marker 240. Both markers 240 and 260 are unique structures and appear in the open space. In general, chemical structures having an identical data encodement profile in high dimensional space, e.g., 512-point dimensional space, will occur as substantially coincident markers on the map. However, in certain situations, markers corresponding to chemical structures having an identical data encodement profile in high dimensional space may appear as fully resolved markers, thereby giving the indication that the markers correspond to nonidentical chemical structures. While not wishing to be bound by theory, it is believed that the stochastic nature of the dimensionality reduction can result in the manifestation of the phenomena described above. The discrete markers corresponding to the query and modified query chemical structures are preferably differentiable so that a user can readily determine when a query chemical structure is identical to and therefore overlaps with an original structure, e.g., 235, or when a query chemical structure is unique with respect to an original structure, e.g., 240. Differentiable discrete markers corresponding to unique structures appear in the open space of the map. Additionally, the differentiable discrete markers allow a user to visualize and evaluate the proximity (and therefore the relatedness) of a modified query chemical structure, e.g. 260, to both an original structure or subset of original structures, e.g., 215/235 and its neighbors, and to the query chemical structure from which it is based, e.g., 235 or 240. The map, including discrete markers and differentiable discrete markers, can be displayed as a graphical user interface using a computer software package known in the art, e.g., software available from Microsoft (Bellevue, Wash.), e.g., Excel (preferred applications are those limited to about 32,000 rows); from OmniViz (Maynard, Mass.); or in preferred embodiments, from Spotfire (Somerville, Mass.). Alternatively, the map can be displayed on graph paper. The differentiability of the markers may also be indicated by using other differentiable means, e.g., color; shading; distinguishable shapes, e.g., squares or circles; or highlighting of the marker border.

Any map described herein can include “add-on” visual output, e.g., one or more additional dimensions of visual output, e.g., the output can specify which of a collection of mapped chemical structures exhibit a common property associated with a particular data field, e.g., patent assignee. For example, a user may wish to know which of the chemical structures are owned by one particular entity, e.g., a corporation or academic institution. This additional output can provide a user with data that supplements or augments the structural relatedness information that is already provided by the map. Further, such output can be desirable especially when it corresponds to properties that inherently may be decoupled or unrelated to structural relatedness, e.g., inventor, year of patenting.

In one embodiment, the markers on the map corresponding to the structures having the common property can be identified using one of the differentiating means described above.

In another embodiment, the structures having the common property can be conveyed to the viewer via a three-dimensional map. In a preferred embodiment, the three dimensional map is a topograhically textured map. Individual structures and/or clusters of two or more structures can be represented as peaks, wherein the height of a particular peak is proportional to the number of structures that occur within the two dimensional area defined by the perimeter of the base of the peak. One advantage of the topographical map is that it can allow a user to readily quantify, in a relative fashion, the populations of one or more visually similar, two-dimensional clusters of markers. This information can therefore assist a user in identifying, e.g., by high density areas, structures having a particular property of interest.

In a preferred embodiment, a three dimensional map can be generated without further computation, i.e., without iterative refinement of the inter-marker distances, from a two dimensional map obtained upon performing a dimensionality reduction, e.g., MDS. In general, the steps include: (i) determining a “term or topic layer,” i.e., the distributed contribution of a single term/topic, e.g., a common property associated with a particular data field, for a collection of mapped chemical structures, e.g., a DOCS, a DOCS+a DQCS; and (ii) applying a smoothing filter to generate the topographical representation. Three-dimensional map generation is known in the art and is described in, e.g., U.S. Pat. No. 6,298,174 B1.

The methods described herein can include, but are not limited to, one or more of the steps shown in FIG. 1, e.g., 205, 225, 245, 250, and 255.

In one embodiment, a method includes mapping a database of chemical structures in which all, i.e., 100 percent, of the structures in the DCS depict chemical entities that are explicitly and/or implicitly disclosed and/or claimed in one or more patent literature documents. In one embodiment, the DCS may include structures belonging to one or more company's patent portfolio. The discrete markers on e.g., map 210 may be color-coded to indicate which structures are associated with a particular company. In certain embodiments, a grid is superimposed onto the map, and the number of structures owned by one company within any grid space may be determined. This calculation may provide a user with the percent “chemical space” owned by a particular company.

In another method, a DOCS and a DQCS can be provided and/or mapped as shown in steps 205 and 225. In certain embodiments, two separate maps may be generated, e.g., a first mapping the DOCS and a second mapping the DQCS relative to the DOCS of the first map, but not necessarily showing explicitly the markers corresponding to the DOCS. For example, the first map may correspond to map 210 in FIG. 1, and the second map may correspond to map 230, but only show markers 235 and 240. In such instances, a frame of reference (e.g., known representative compound set) between the two maps must be established in order for a meaningful comparison to be obtained. The first may be overlaid with the second to determine the extent of similarity between the two databases. Structures from the DQCS map that appear in the white space of the DOCS map are unique structures with respect to the DOCS. Alternatively, the map (e.g., 210) may automatically be updated, e.g., in response to the single depression and release of an input device (e.g., a computer mouse button, its equivalent on a laptop computer, keyboard keystroke, etc.), with the differentiable discrete markers corresponding to the query chemical structures to provide, e.g., map 215. Unique structures appear in the open space. The above method can be carried out using a DQCS having exclusively de novo structures, e.g., structures obtained from a proprietary database. In each of the above cases, if all of the structures in the DOCS depict chemical entities that are explicitly and/or implicitly disclosed and/or claimed in one or more patent literature documents, then the above method can allow a user to rapidly identify structures that fall within the exploratory space. A user can compare a unique, i.e. an exploratory space, DQCS structure with e.g., a nearest neighbor DOCS structure, to determine what structural attributes of the DQCS structure distinguish it from the DOCS structure. This observation can provide a user with insight as to how to identify and generate other unique structures. Repeating the above steps in an iterative fashion can ultimately provide a user with information as to the structural “nature” of this exploratory space.

In another method, a DOCS and a DQCS can be provided and/or mapped as shown in steps 205 and 225 and in maps 210 and 230. The query chemical structure corresponding to differentiable discrete marker 240 is unique with respect to the DOCS because it appears in the open space. If the user's objective is to identify a structure that is increasingly dissimilar to 215 (and its neighbors), i.e. more dissimilar than is 240, then the following steps may be carried out. A modified query chemical structure based on 240 can be generated using, e.g., a compound enumeration method known in the art. For example, a user may enumerate structures using a computer program, e.g., SPROUT, which builds structures in a stepwise manner by (1) generating skeletons or molecular graphs that satisfy steric constraints and (2) converting the skeletons to molecules by making atom substitutions. The SPROUT program is described in, e.g., Gillet, et al J. Chem. Inf. Comput. Sci. 1994, 34, 207 and references therein. Structure enumeration may also be performed with, e.g., simulated annealing, which is an algorithm based on the analogy between the simulated annealing of solids and large scale optimization problems, or a genetic algorithm, which is a computational analog of Darwinian evolution that can produce new individual examples, e.g., chemical structures, from combinations of previous examples. In one embodiment, a genetic algorithm can be used in concert with a database that encodes a plurality of combinatorial reactions (e.g., from Sertanty, San Jose, Calif. www.info@sertanty.com) to evolve libraries of open space DMQCS structures that occur at a specific distance, e.g., a minimum distance or a selected distance, from a particular DOCS or DQCS structure. These enumeration methods are described in Faulon et al. J. Chem. Inf. Comput. Sci. 2003, 43, 721 and references therein; Gillet et al. J. Chem. Inf. Comput. Sci. 2002, 42, 375 and references therein; Sheridan et al. J. Chem. Inf. Comput. Sci. 1995, 35, 310 and references therein; Kvasnicka et al. J. Chem. Inf. Comput. Sci. 1996, 36, 516 and references therein; and Meiler et al. J. Chem. Inf. Comput. Sci. 2001, 41, 1535 and references therein. The enumeration methods can assist the user in identifying how to modify 240 so as to create a new query chemical structure that is more dissimilar to 215 than 240 is to 215. With a modified query chemical structure in hand; step 250 may be carried out generating map 265, which includes 260, a more dissimilar structure (relative to 215) than 240. If identical marker 235 had been selected as the basis of the modified query chemical structure, then steps 250 and 255 could be iteratively repeated with different modified query chemical structures until a unique structure is found. The steps could be repeated using, e.g., the same query chemical structure and a different modification, a different query chemical structure and the same modification, or a different query chemical structure and different modification. Once a unique structure is found, the same steps could be again repeated until a particular similarity/dissimilarity criterion is met. Thus, the method can provide a user with a rapid method for identifying de novo structures. Again, when the DOCS is based exclusively on structures from the patent literature, these techniques can allow a user to efficiently identify unique structures that are increasingly “potentially novel” or further into a particular exploratory space (e.g., 260).

The above methods may be further incorporated into the drug discovery process.

For example, a de novo structure identified by one of the above methods may be subjected to in silico, e.g., computer-aided drug design methods, using proprietary or commercially available software packages for correlating structural descriptors or quantitative structure-activity relationships (QSAR). These programs use efficacy data for previously tested compounds to predict the efficacy of compounds yet to be tested and can provide a user with an accurate prediction as to the activity of a compound before it is tested (or even synthesized). Exemplary software packages can include, e.g., Comparative Molecular Field Analysis (CoMFA, Tripos, Inc., St. Louis, Mo.) and HQSAR (Tripos, Inc., St. Louis, Mo.). CoFMA uses variance in field strengths around a set of aligned structures, e.g., three-dimensional structures to describe the observed variance in biological activity. In the HQSAR program, structures are divided into all possible connectivity atom-bond fragments of predetermined size (e.g., number of atoms). Once the descriptors have been identified, statistical methods generate a QSAR model relating descriptors to activity.

A user of one of the above methods may also wish to synthesize one or more compounds corresponding to one or more de novo structures identified by any of the above methods described herein. The user may perform the synthesis or instruct a skilled artisan in organic synthesis to prepare the compounds by conventional or automated methods. Synthetic chemistry transformations and protecting group methodologies (protection and deprotection) useful in synthesizing the de novo compounds obtained by methods described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The de novo compounds obtained by methods described herein can be separated from a reaction mixture and further purified, e.g., for in vivo or in vitro testing, by a method such as column chromatography, high-pressure liquid chromatography, or recrystallization. The compounds may be isolated and/or tested as a salt or prodrug thereof.

In an alternate embodiment, the de novo compounds obtained by methods described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60. Thus, one embodiment relates to a method of using the compounds identified by methods described herein (e.g., unique chemical structures, modified query chemical structures) for generating derivatives or chemical libraries comprising: 1) providing a body comprising a plurality of wells; 2) providing one or more compounds identified by methods described herein (e.g., unique chemical structures, modified query chemical structures) in each well; 3) providing an additional one or more chemicals in each well; 4) isolating the resulting one or more products from each well. An alternate embodiment relates to a method of using the compounds identified by methods described herein (e.g., unique chemical structures, modified query chemical structures) for generating derivatives or chemical libraries comprising: 1) providing one or more compounds identified by methods described herein (e.g., unique chemical structures, modified query chemical structures) attached to a solid support; 2) treating the one or more compounds identified by methods described herein (e.g., unique chemical structures, modified query chemical structures) attached to a solid support with one or more additional chemicals; 3) isolating the resulting one or more products from the solid support. In the methods described above, “tags” or identifier or labeling moieties may be attached to and/or detached from the compounds identified by methods described herein (e.g., unique chemical structures, modified query chemical structures) or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

A user of one of the above methods may also wish to test one or more compounds corresponding to one or more unique structures or derivatives of unique structures, e.g. de novo structures identified by any of the above methods described herein using an in vitro or in vivo screening method. For example, a library described above may been tested using a High Throughput Screening (HTS) method, e.g., placing compounds in micro titre plates and contacting the compound with a biological receptor. The receptor may be isolated or a cell line may be engineered to give a detectable response when the receptor is modulated by the compound. Individual compounds or smaller groups of compounds, e.g., 2-95 compounds, may be tested in a similar fashion. Active compounds may be further analyzed according to an FDA-recognized battery of ADME studies, e.g., absorption, disposition, metabolism and excretion. The compounds may be further evaluated in animal toxicolgy studies, and ultimately, in human clinical trials. Finally any de novo compound maybe the subject of structure-activity relationship studies (SAR), which, in turn, may result in the above steps being repeated with analogs of the de novo compound.

The methods of the invention can be implemented in digital electronic circuitry, or in computer hardware, firmware, software, or in combinations thereof. Computer assistance allows powerful manipulations of chemical structural data and permits automation. Furthermore, computer assistance makes possible the simultaneous comparision and recombination of multiple molecules. According to an embodiment of the invention, an apparatus (e.g., a computer), can contain computer instructions and systems that effect mapping of chemical structures. The instructions and systems can can be implemented in a computer program product tangibly embodied in a machine-readable storage device for execution by a programmable processor; and method actions can be performed by a programmable processor executing the instructions to perform chemical structure mapping by operating on input data and generating output.

The steps of the modeling methods can include both steps implemented by commercially available software packages, and steps implemented by instructions provided by a scripting language (e.g., Perl, Python), or a compiled language (e.g., C, Fortran). Also, the steps can be integrated using instructions provided with a computer language, such as those mentioned above.

The methods and systems of the invention can be implemented advantageously in one or more computer programs that are executable on a programmable system including at least one programmable processor coupled to receive data and instructions from, and to transmit data and instructions to, a data storage system, at least one input device, and at least one output device. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks and removable disks; magneto-optical disks; and CD_ROM disks. Any of the foregoing can be supplemented by, or incorporated in, ASICs (application-specific integrated circuits).

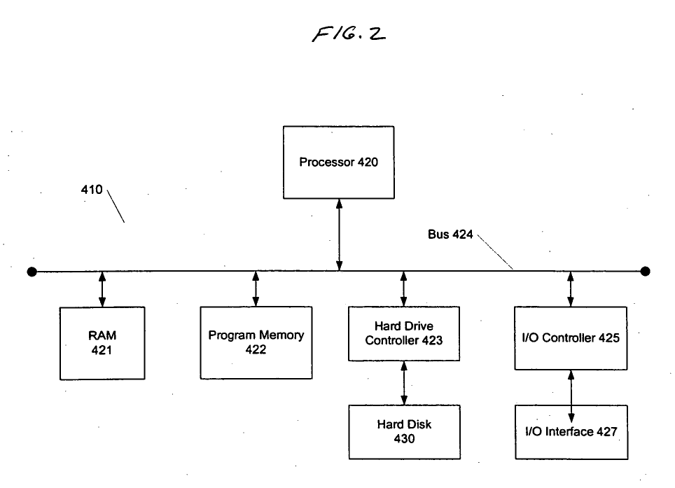

An example of one such type of computer is shown in FIG. 2, which shows a block diagram of a programmable processing system (system) 410 suitable for implementing or performing the apparatus or methods of the invention. The system 410 includes a processor 420, a random access memory (RAM) 421, a program memory 422 (for example, a writable read-only memory (ROM) such as a flash ROM), a hard drive controller 423, and an input/output (I/O) controller 425 coupled by a processor (CPU) bus 424. The system 410 can be preprogrammed, in ROM, for example, or it can be programmed (and reprogrammed) by loading a program from another source (for example, from a floppy disk, a CD-ROM, or another computer).

The hard drive controller 423 is coupled to a hard disk 430 suitable for storing executable computer programs, including programs embodying the present invention, and data including storage. The I/O controller 425 is coupled by means of an I/O bus 424 to an I/O interface 427, that can include one or more of the following: a monitor, a mouse, a keyboard or other input device. The I/O interface 427 receives and transmits data in analog or digital form over communication links such as a serial link, local area network, wireless link, and parallel link.

One non-limiting example of an execution environment includes computers running Windows NT 4.0 (Microsoft) or Linux operating systems. Browsers can be Microsoft Internet Explorer version 4.0 or greater or Netscape Navigator or Communicator version 4.0 or greater. Computers for databases and administration servers can include Windows NT 4.0 with a 400 MHz Pentium II (Intel) processor or equivalent using 256 MB memory and 9 GB SCSI drive. Computer Node Hosts can include Windows NT 4.0 with a 400 MHz Pentium II (Intel) processor or equivalent using 128 MB memory and 5 GB SCSI drive. Other environments could of course be used.

### EXAMPLES

### Example 1

### Mapping a Database of Chemical Structures

1. SMILES representations were created for all exemplified structures, i.e. explicitly disclosed, in the following patents: WO/93/20066 (Merck—19 structures, PAT_ID1); WO/02/094814 (Schering—21 structures, PAT_ID2); WO/00/17159 (Tularik—22 structures, PAT_ID3); WO 0164653 (Astra Zeneca—25 structures, PAT_ID4) using the following procedure: (i) the structures were sketched into ChemDraw 6.0 (CambridgeSoft, Cambridge, Mass.); (ii) the sketched structures were highlighted; (iii) the highlighted structures were copied using the “Copy As SMILES” command from the Edit menu; and (iv) the SMILES string was pasted into a text file.

2. Fingerprint representations of each molecule were created using the “fingerprint” program from Daylight Chemical Information Systems (Mission Viejo, Calif.) and the text file created in step 1. The maximum and minimum number of allowed bits in the fingerprint were set to 512.

3. The statistics package R (Ihaka, R., Gentleman, R, (1996), “R: A Language for Data Analysis and Graphics”, Journal of Computational and Graphical Statistics, 5, 299-314, http://www.r-project.org, version 1.6.1) was used to perform a principal components analysis on the fingerprint file created in step 2.

4. The molecule identifier and the first two associated principal components for each fingerprint were saved to a text file.

5. The rotation matrix produced by the principal components analysis was saved to a text file.

6. The appropriate patent assignee identifier for each molecule record was appended to the text file created in step 5. The resulting text file was saved.

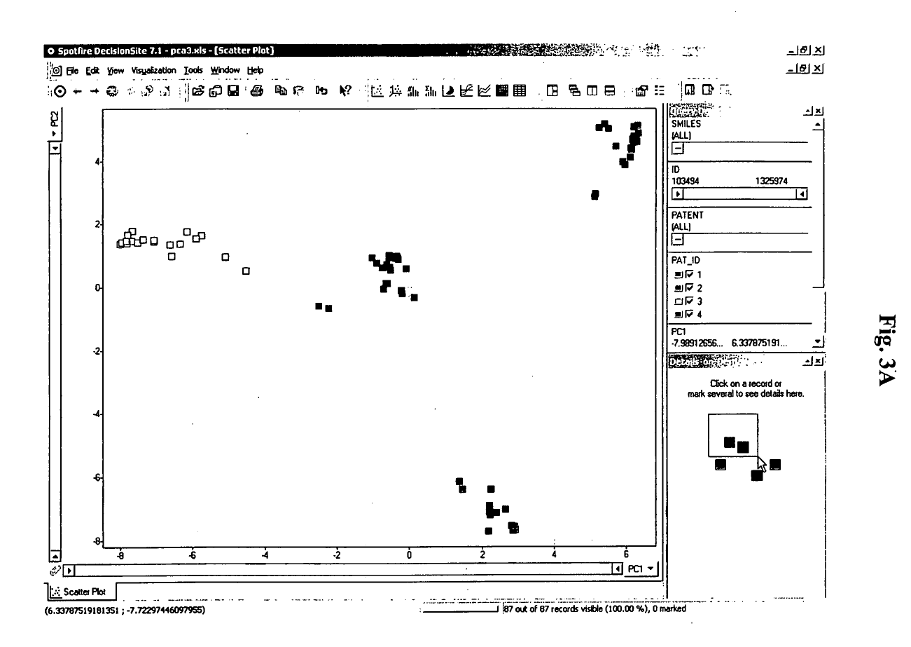

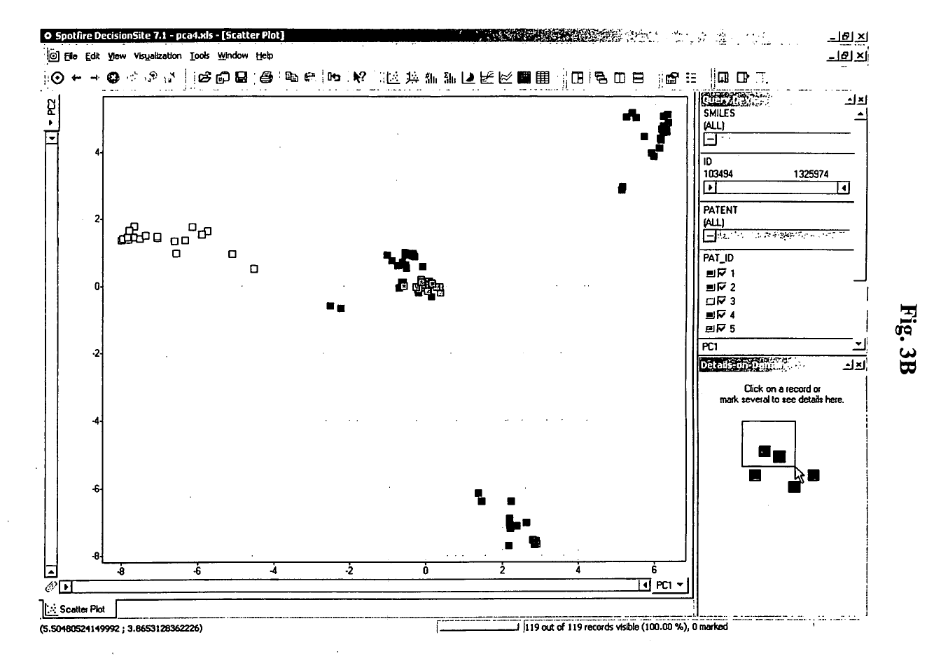

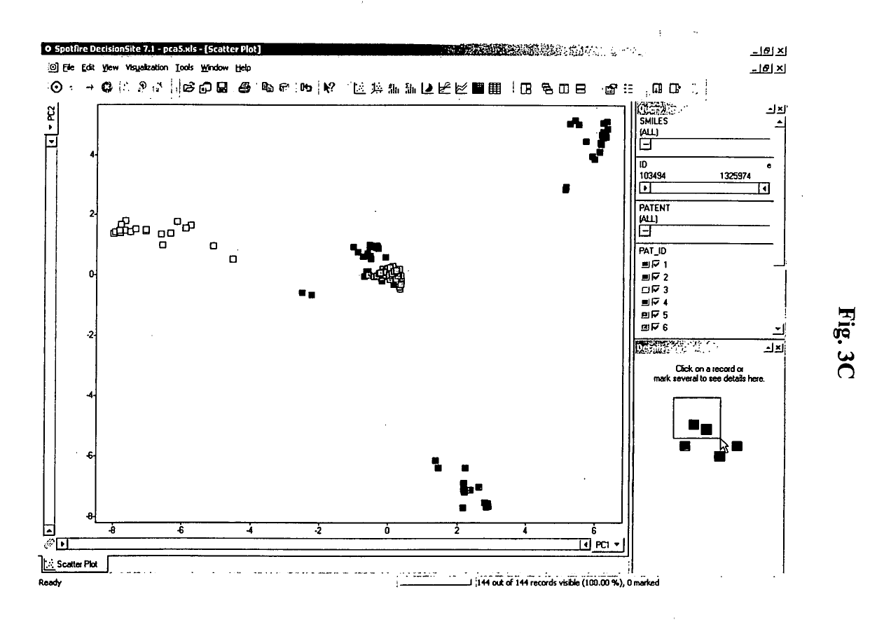

7. The text file created in step 5 was visualized using Spotfire (Spotfire, Inc, Somerville, Mass.). The first principal component is displayed on the x axis, the second principal component is displayed on the y axis. The discrete markers were further color-coded according to the entry in the patent assignee data field. The map is shown as FIG. 3A, and clearly shows that each set of commonly owned chemical structures, i.e., the red (PAT_ID1), blue (PAT_ID2), yellow (PAT_ID3), and black (PAT_ID4) markers, are more structurally similar to one another than they are to the structures owned by the other entities, e.g., the yellow markers are closer to one another than they are to any of the black, red or blue markers. One could conclude that each entity is in possession of structurally dissimilar patented compound portfolios.

The map generated above is used as the DOCS for the subsequent examples below.

### Example 2

### Adding a Database of Query Chemical Structures (DQCS) to an Existing Map (Adding a DOCS to a Database of Original Structures (DOCS)

1. The method described in Example 1, step 1 was used to create SMILES representations for all exemplified structures in the following patent: WO0138311 (Glaxo —32 structures, PAT_ID5).

2. Fingerprint representations of each molecule were created using the method described in Example 1, step 2.

3. The fingerprint file created in step 2 was read into the statistics package R, this operation created matrix A (i.e., a DQCS).

4. The rotation matrix created in step 5 of Example 1 was read into the statistics package R, this operation created matrix B (i.e., a DOCS).

5. Statistics package R was used to perform a matrix multiplication on matrices A and B to create matrix C.

6. The first molecule identifier and first two columns of matrix C were saved to a file.

7. The appropriate patent identifier for each molecule record was appended to the text file created in step 6. This text file was saved.

8. The file created in step 7 was appended to the file created in step 6 of Example 1.

9. The text file created in step 8 was visualized as described in Example 1, step 7. The resulting map is shown as FIG. 3B. The yellow, black, red and blue diescrete markers represent the structures corresponding to the DOCS. The green differentiable discrete markers correspond to the structures corresponding to the DQCS (PAT_ID 5). The DQCS structures are unique with respect to the DOCS structures as they fall within the white space of the map generated in Example 1. The DQCS structures also overlap both closely with one another and with the black markers, i.e., corresponding to (PAT_ID4). One could conclude that the owners of the “green” structures may be working in a similar therapeutic area as the owners of the “black” structures.

### Example 3

### Adding a Database of Query Chemical Structures Obtained From a Combinatorial Library Enumeration Method (DQCS) to an Existing Map

1. A virtual combinatorial library of 25 compounds, e.g., potential de novo compounds, based on the quinazoline chemistry shown below was enumerated. 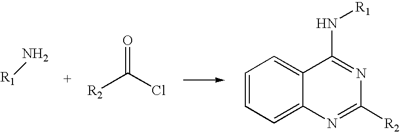
Library enumeration was performed using a computer program which makes use of the Reaction Toolkit from Daylight Chemical Information Systems (Mission Viejo, Calif.). A set of 5 primary amines
CC(═O)OCC(N)C(═O)O
CC(C(N)C(═O)O)clcccccl
CC(C)(C)C(N)C(═O)O
CC(C)(C)ClCCC(CCl)N
CC(C)(C)CC(C)(C)N
and 5 acid chlorides
CC(═O)Cl
CCC(═O)Cl
ClC(═O)CiCCl
CC(C)C(═O)Cl
CCCC(═O)Cl
were used to generate the virtual library (PAT_ID6).

2. A fingerprint representation of each molecule was created using the method described in Example 1, step 2.

3. The fingerprint file created in step 2 was read into the statistics package R, this operation will create matrix A (e.g., a DQCS).

4. The rotation matrix created in step 5 of Example 1 was read into the statistics package R, this operation created matrix B (DOCS).

5. Statistics package R was used to perform a matrix multiplication on matrices A and B to create matrix C.

6. The first molecule identifier and first two columns of matrix C was saved to a file.

7. The appropriate patent identifier for each molecule record was appended to the text file created in step 6. This text file was saved.

8. The file created in step 7 was appended to the file created in step 6 of Example 1.

9. The text file created in step 8 was visualized as described in Example 1, step 7.

The resulting map is shown as FIG. 3C. The yellow, black, red and blue diescrete markers represent the structures corresponding to the DOCS. The green differentiable discrete markers correspond to the structures corresponding to the DQCS (PAT_ID 5). The turquoise differentiable discrete markers correspond to the structures corresponding to the DMQCS (PAT_ID6). The DQCS structures that fall within the white space on the map are unique with respect to the DOCS and the DQCS. These structures therefore represent explorable and potential de novo and compounds or scaffolds. The DQCS structures also overlap both closely with one another and with the black and green markers, i.e., corresponding to (PAT_ID4) and (PAT_ID5).

### Example 4

### Mapping Compounds to Monitor Competitor Intelligence

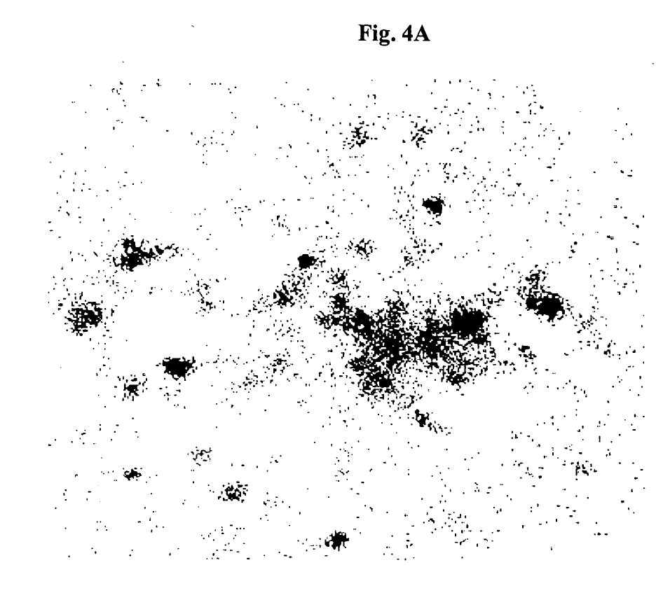

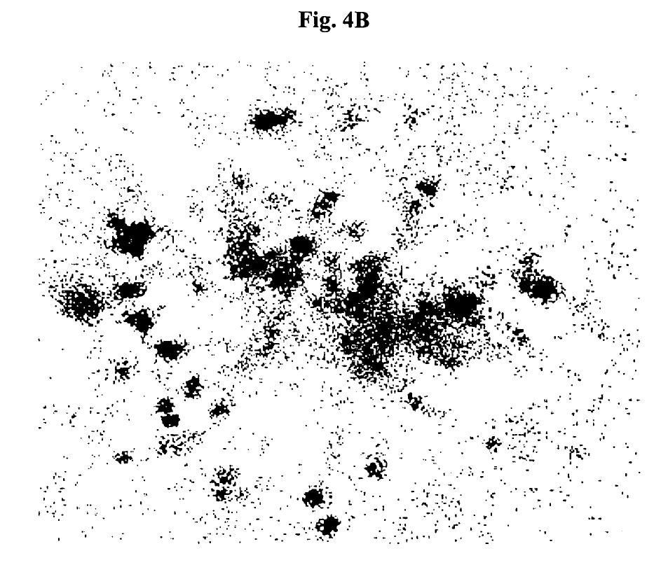

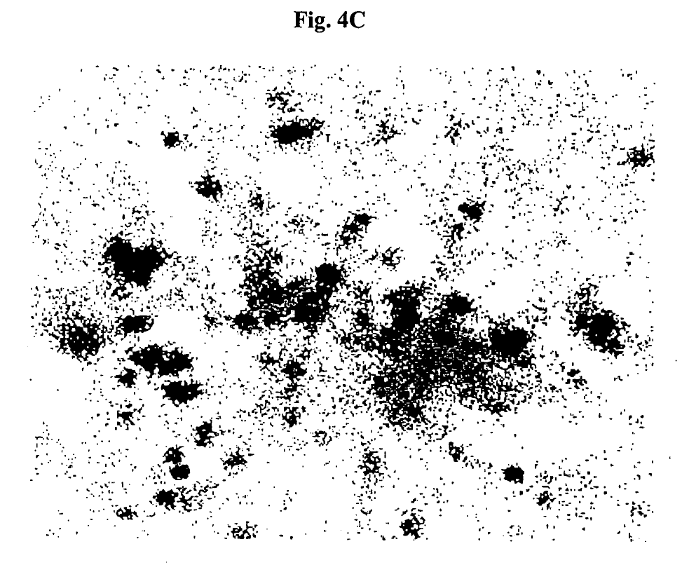

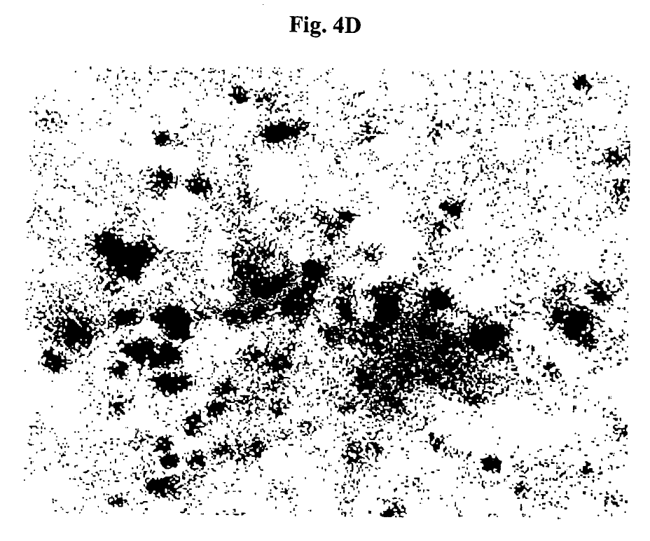

FIG. 4A shows a map generated as described in Examples 1-3. The discrete markers on the map correspond to structures of compounds that are covered by patents issued over the course of one year (1999) that are assigned to one of three different entities. The markers are color-coded to distinguish among the three entities (red=entity 1; blue=entity 2; and green=entity 3).

FIGS. 4B, 4C, and 4D show changes over three sequential years (2000-2002) in the map shown in FIG. 4A. Each of the maps was generated by updating the original map shown in FIG. 4A with new chemical structures. The maps point out, e.g., that entities 1 and 2 have portfolios of compounds that are similar to one as judged by the proximities of the red and blue clusters on the map. The entity 3 portfolio initially consists of several groups of compounds that are both dissimilar to one another and to the entity 1 and 2 compounds. It is clear that over time entity 3 began to patent compounds that are similar to entity 1 and 2 compounds as judged by the new cluster of green markers in proximity to the red and blue clusters on the map.

All references cited herein, whether in print, electronic, computer readable storage media or other form are expressly incorporated by reference in their entirety, including but not limited to, abstracts, articles, journals, publications, texts, treatises, internet websites, databases, patents and patent publications.

A number of embodiments of the invention have been described. Nevertheless, it will be understood that various modifications may be made without departing from the spirit and scope of the invention. Accordingly, other embodiments are within the scope of the following claims.

1. A method for visualizing a database of chemical structures from the patent literature, the method comprising mapping a database of chemical structures from patent literature documents, wherein each of the chemical structures is displayed on a map as a discrete marker, and the intervening space between the discrete markers is displayed on the map as a continuum that visually contrasts with the discrete markers.

2. The method of claim 1, wherein the structures are explicitly disclosed and/or claimed in U.S. patents and/or U.S. patent application publications.

3. The method of claim 1, wherein the structures are explicitly disclosed and/or 1 claimed in PCT application publications.

4. The method of claim 1, wherein the structures are explicitly disclosed and/or claimed in non-U.S. patents and/or non-U.S. patent application publications.

5. The method of claim 1, wherein the structures are implicitly disclosed and/or claimed in U.S. patents and/or U.S. patent application publications.

6. The method of claim 1, wherein the structures are implicitly disclosed and/or claimed in PCT application publications.

7. The method of claim 1, wherein the structures are implicitly disclosed and/or claimed in non-U.S. patents and/or non-U.S. patent application publications.

8. The method of claim 1, wherein the database of chemical structures further includes one or more data fields related to each of the chemical structures.

9. The method of claim 8, wherein the data field is biological assay data related to one or more biological targets.

10. The method of claim 8, wherein the data field is a medical indication.

11. The method of claim 8, wherein the data field is a physical property.

12. The method of claim 8, wherein the data field is a key word.

13. The method of claim 8, wherein the data field is a patent assignee.

14. The method of claim 8, wherein the data field is a patent issue date.

15. The method of claim 8, wherein the data field is a patent application filing date.

16. The method of claim 1, wherein the map is a non-linear map.

17. The method of claim 16, wherein the distance between any two discrete markers on the map is representative of the similarity or dissimilarity between the corresponding chemical structures.

18. The method of claim 1, wherein the mapping is carried out according to a user-defined similarity parameter.

19. The method of claim 1, wherein the user-defined similarity parameter is structural similarity.

20. The method of claim 1, wherein the map is a linear map.

21. The method of claim 8, wherein the data field is inventor name.

22. The method of claim 8, wherein the data field is inventory data.

23. A method for generating a database of compounds that are outside of an original database, the method comprising:
(a) mapping a database of original chemical structures, wherein each of the original chemical structures is displayed as a discrete marker, and the intervening space between the discrete markers is displayed as a continuum that visually contrasts with discrete markers;
(b) mapping a database of query chemical structures, wherein each of the query chemical structures is displayed as a differentiable discrete marker that is differentiable from the discrete markers corresponding to the database of original chemical structures; and
(c) determining the degree of similarity between the query chemical structures and the original chemical structures.

24. The method of claim 23, wherein the original chemical structures are from patent literature documents.

25. The method of claim 24, wherein the structures are explicitly or implicitly disclosed and/or claimed in U.S. patents and/or U.S. patent application publications.

26. The method of claim 24, wherein the structures are explicitly or implicitly disclosed and/or claimed in published PCT applications.

27. The method of claim 24, wherein the structures are explicitly or implicitly disclosed and/or claimed in non-U.S. patents and/or non-U.S. patent application publications.

28. The method of claim 24, wherein the database of original chemical structures is further provided with one or more data fields related to each of the original chemical structures.

29. The method of claim 28, wherein the data field is biological assay data related to a particular target.

30. The method of claim 28, wherein the data field is a medical indication.

31. The method of claim 28, wherein the data field is a physical property.

32. The method of claim 28, wherein the data field is a key word.

33. The method of claim 28, wherein the data field is a patent assignee.

34. The method of claim 28, wherein the data field is a patent issue date.

35. The method of claim 28, wherein the data field is a patent application filing date.

36. The method of claim 23, wherein steps (a) and (b) are performed simultaneously.

37. The method of claim 36, wherein the discrete markers corresponding to the original chemical structures, the intervening space between the discrete markers, and the differentiable discrete markers corresponding to the query chemical structures are displayed on a map.

38. The method of claim 37, wherein the map is a non-linear map.

39. The method of claim 38, wherein the distance between any two discrete markers on the map is representative of the similarity or dissimilarity between the corresponding chemical structures.

40. The method of claim 23, wherein steps (a) and (b) are performed at different times.

41. The method of claim 40 further comprising:
(i) displaying the discrete markers corresponding to the original chemical structures and the intervening space between the discrete markers on a first map;
(ii) displaying the differentiable discrete markers corresponding to the query chemical structures on a second map; and
(iii) overlaying the first and second maps.

42. The method of claim 41, wherein each map is a non-linear map.

43. The method of claim 42, wherein the distance between any two discrete markers on each map and on the overlay of the two maps is representative of the similarity or dissimilarity between the corresponding chemical structures.

44. The method of claim 40 further comprising displaying the discrete markers corresponding to the original chemical structures and the intervening space between the discrete markers on a map that is automatically updated with the differentiable discrete markers corresponding to the query chemical structures once step (b) is performed.

45. The method of claim 44, wherein the map is a non-linear map.

46. The method of claim 45, wherein the distance between any two discrete markers on the map is representative of the similarity or dissimilarity between the corresponding chemical structures.

47. The method of claim 23, wherein the mapping is carried out according to a according to a user-defined similarity parameter.

48. The method of claim 23, wherein the user-defined similarity parameter is structural similarity.

49. The method of claim 44, wherein the map is a linear map.

50. The method claim 23, wherein the query chemical structure is a unique query chemical structure.

51. The method of claim 50, wherein the unique query chemical structures are de novo structures.

52. The method of claim 23, wherein the method further comprises the step of providing a database of original chemical structures structures.

53. The method of claim 52, wherein the providing step further comprises representing the chemical structures in binary form or in the form of binary fingerprints.

54. The method of claim 23, wherein the method further comprises the step of providing a database of query chemical structures.

55. The method of claim 54, wherein the providing step further comprises representing the chemical structures in binary form or in the form of binary fingerprints.

56. A method for generating a database of compounds that are outside of an original database, the method comprising:
(a) providing a database of original chemical structures;
(b) mapping the database of original chemical structures, wherein each of the chemical structures is displayed on a map as a discrete marker and the intervening space between the markers is displayed on the map as a continuum that visually contrasts with the plurality of discrete markers;
(c) providing a database of one or more query chemical structures;
(d) mapping the database of query chemical structures, wherein each query chemical structure is displayed on the map from step (b) as a differentiable discrete marker that is differentiable from the discrete markers corresponding to the original chemical structures;
(e) determining the degree of similarity between the query chemical structures and the original chemical structures;
(f) providing a database of one or more modified query chemical structures, wherein each structure corresponds to a query chemical structure from step (c) having a modification, and wherein the modification is chosen so that the modified query chemical structure is less similar to a comparative subset of original chemical structures than the query chemical structure before the modification;
(g) mapping the database of modified query chemical structures, wherein each modified query structure is displayed on the map from step (b) or step (d) as a differentiable discrete marker that is differentiable from the discrete markers corresponding to the original chemical structures and from the differentiable discrete markers corresponding to the query chemical structures; and
(h) determining the degree of similarity between the modified query chemical structures and the comparative subset of original chemical structures from step (f).

57. The method of claim 56, wherein steps (c)-(e) are repeated until a query chemical structure is found that is unique with respect to the original chemical structures.

58. The method of claim 56, wherein steps (f)-(h) are performed on a query chemical structure that substantially similar to an original chemical structure.

59. The method of claim 56, wherein steps (f)-(h) are repeated.

60. The method of claim 59, wherein steps (f)-(h) are repeated using the same query chemical structure and a different modification.

61. The method of claim 59, wherein steps (f)-(h) are repeated using a different query chemical structure and the same modification.

62. The method of claim 59, wherein steps (f)-(h) are repeated using a different query chemical structure and a different modification.

63. The method of claim 59, wherein the original chemical structures are from the patent literature documents.

64. The method of claim 63, wherein the structures are explicitly or implicitly disclosed and/or claimed in U.S. patents and/or U.S. patent application publications.

65. The method of claim 63, wherein the structures are explicitly or implicitly disclosed and/or claimed in PCT application publications.

66. The method of claim 63, wherein the structures are explicitly or implicitly disclosed and/or claimed in non-U.S. patents and/or non-U.S. patent application publications.

67. The method of claim 56, wherein steps (a), (c), and (f) further include representing the chemical structures in binary form.

68. The method of claim 56, wherein steps (a), (c), and (f) further include representing the chemical structures in the form of binary fingerprints.

69. The method of claim 56, wherein the database of original chemical structures is further provided with one or more data fields related to each of the original chemical structures.

70. The method of claim 69, wherein the data field is biological assay data related to a particular target.

71. The method of claim 69, wherein the data field is a medical indication.

72. The method of claim 69, wherein the data field is a physical property.

73. The method of claim 69, wherein the data field is a key word.

74. The method of claim 69, wherein the data field is a patent assignee.

75. The method of claim 69, wherein the data field is a patent issue date.

76. The method of claim 69, wherein the data field is a patent application filing date.

77. The method of claim 56, wherein the map is a non-linear map.

78. The method of claim 77, wherein the distance between any two discrete markers on the map is representative of the similarity or dissimilarity between the corresponding chemical structures.

79. The method of claim 56, wherein the mapping is carried out according to a user-defined similarity parameter.

80. The method of claim 56, wherein the user-defined similarity parameter is structural similarity.

81. The method of claim 56, wherein the map is a linear map.

82. The method of claim 56, wherein the query chemical structure is a unique query chemical structure.

83. A method for generating a database of compounds that are outside of an original database, the method comprising:
(a) providing a database of original chemical structures;
(b) mapping the database of original chemical structures, wherein each of the chemical structures is displayed on a map as a discrete marker and the intervening space between the markers is displayed on the map as a continuum that visually contrasts with the plurality of discrete markers;
(c) providing a database of one or more de novo chemical structures;
(d) mapping the database of de novo chemical structures, wherein each de novo chemical structure is displayed on the map from step (b) as a differentiable discrete marker that is differentiable from the discrete markers corresponding to the original chemical structures;
(e) determining the degree of similarity between the de novo chemical structures and the original chemical structures; and
(f) evaluating the number of discrete markers in the intervening space continuum.

84. A database generated by:
(a) providing a database of original chemical structures;
(b) mapping the database of original chemical structures, wherein each of the chemical structures is displayed on a map as a discrete marker and the intervening space between the markers is displayed on the map as a continuum that visually contrasts with the plurality of discrete markers;
(c) providing a database of one or more unique query chemical structures, wherein each unique query chemical structure is unique with respect to the original chemical structures;
(d) mapping the database of unique query chemical structures, wherein each unique chemical structure is displayed within the intervening space continuum on the map from step (b) as a differentiable discrete marker that is differentiable from the discrete markers corresponding to the original chemical structures;
(e) determining the degree of similarity between the unique query chemical structures and the original chemical structures;
(f) providing a database of one or more modified unique query chemical structures, wherein each modified structure corresponds to a unique query chemical structure from step (c) having a modification, and wherein the modification is chosen so that the modified unique query chemical structure is less similar to a comparative subset of original chemical structures than the unique query chemical structure was before the modification;
(g) mapping the modified unique query chemical structures, wherein each modified unique query chemical structure is displayed on the map from step (b) or step (d) as a differentiable discrete marker that is differentiable from the discrete markers corresponding to the original chemical structures and from the discrete markers corresponding to the unique query chemical structures; and
(h) determining the degree of similarity between the modified unique query chemical structures and the comparative subset of original chemical structures from step (f).

85. A method for designing a drug candidate, the method comprising:
(a) providing a database of original chemical structures;
(b) mapping the database of original chemical structures, wherein each of the chemical structures is displayed on a map as a discrete marker and the intervening space between the markers is displayed on the map as a continuum that visually contrasts with the plurality of discrete markers;
(c) providing a database of one or more de novo chemical structures;
(d) mapping the database of de novo chemical structures, wherein each de novo chemical structure is displayed on the map from step (b) as a differentiable discrete marker that is differentiable from the discrete markers corresponding to the original chemical structures;
(e) determining the degree of similarity between the de novo chemical structures and the original chemical structures;
(f) evaluating the number of differentiable discrete markers located in the intervening space continuum.
(g) selecting a chemical structure corresponding to a differentiable discrete marker located in the intervening space continuum; and
(h) subjecting the chemical structure to computer-aided drug design methods.

86. The method of claim 85, wherein the method further comprises synthesizing the compound corresponding to the structure selected in step (g).

87. The method of claim 85, wherein the method further comprises evaluating the compound's ability to modulate a target through in vivo and/or in vitro methods.

88. A method for visualizing the relationship of a drug candidate chemical structure to structures in a database, the method comprising:
(a) providing a database of original chemical structures;
(b) mapping the database of original chemical structures, wherein each of the chemical structures is displayed on a map as a discrete marker and the intervening space between the markers is displayed on the map as a continuum that visually contrasts with the plurality of discrete markers;
(c) providing a database of one or more drug candidate chemical structures;
(d) mapping the database of drug candidate chemical structures, wherein each drug candidate chemical structure is displayed on the map from step (b) as a differentiable discrete marker that is differentiable from the discrete markers corresponding to the original chemical structures; and
(e) evaluating the number of differentiable discrete markers located in the intervening space continuum.

89. The method of claim 88, wherein the method further comprises:
(f) selecting a drug candidate chemical structure corresponding to a differentiable discrete marker located in the intervening space continuum;
(g) measuring the distance between the differentiable discrete marker selected in step (f) and each of the discrete markers corresponding to the original chemical structures;
(h) determining the discrete marker that is closest in linear distance to the differentiable discrete marker selected in step (f);
(i) comparing the structure corresponding to the discrete marker determined in step (h) with the structure of the drug candidate structure corresponding to the differentiable discrete marker selected in step (f);
(j) determining the discrete marker that is next closest in linear distance to the differentiable discrete marker selected in step (f); and
(k) comparing the structure corresponding to the discrete marker determined in step (j) with the structure of the drug candidate structure corresponding to the differentiable discrete marker selected in step (f).

90. The method of claim 89, wherein steps (j) and (k) are repeated.
